(19) United States
(12) Patent Application Publication
(10) Pub. No.: US 2010/0217178 A1
(43) Pub. Date: Aug. 26, 2010

(54) PREDICTION OF PERITONEAL DIALYSIS THERAPY OUTCOMES USING DIALYSATES MIXED AT DIFFERENT GLUCOSE CONCENTRATIONS

(75) Inventors: Ying-Cheng Lo, Green Oaks, IL (US); Alp Akonur, Evanston, IL (US); Isaac Martis, Chicago, IL (US); Andrew C. Hayes, Libertyville, IL (US)

Correspondence Address: K&L Gates LLP, P.O. Box 1135, Chicago, IL, 60690-1135, US

(73) Assignees: BAXTER INTERNATIONAL INC., Deerfield, IL (US); BAXTER HEALTHCARE S.A., Zurich (CH)

(21) Appl. No.: 12/389,751

(22) Filed: Feb. 20, 2009

(51) Int. Cl. A61M 1/28; G06N 5/02; G06F 17/10; G06G 7/60

(52) U.S. Cl. 604/28; 706/46; 703/2; 703/11

(57) ABSTRACT

A method for peritoneal dialysis treatment includes (i) predicting results of a plurality of patient therapy outcomes for a plurality of different mixed dextrose level dialysis solutions; (ii) selecting one of the mixed dextrose level solutions for a patient based on the results; and (iii) performing at least one therapy using different unmixed dextrose level solutions that combine to simulate a like cumulative concentration that would be achieved using the selected mixed dextrose level solution.

### BACKGROUND

The present disclosure relates to medical fluid delivery and more specifically to peritoneal dialysis (“PD”).

PD fluid called dialysate is provided typically in standard glucose levels. For example, in the United States, dialysate is provided typically in standardized glucose levels of 1.36%, 2.27% and 3.86% (corresponding to dextrose levels of 1.5%, 2.5% and 4.25%, respectively). The higher the glucose level, the higher the osmotic gradient caused by the dialysate, causing a larger amount of ultrafiltrate (“UF”) or waste water to be removed from the patient. The higher the glucose level, however, the more calories provided by the dialysate, and the more weight that can be gained potentially by the patient.

Patients accordingly typically use the lowest glucose level dialysate possible that will remove a needed amount of UF. Sometimes, however, the patient's therapy needs fall in between the standardized glucose levels of 1.36%, 2.27% and 3.86%. It may be desirable to use a blended glucose level of, for example, 2.0% glucose.

Tests can be performed on the patient to see how effective a particular dialysate is at removing waste and UF from the patient. For example, a peritoneal equilibrium test (“PET”) can be performed, which analyzes samples of dialysate taken after different dwell periods within the patient's peritoneum. The PET also requires analysis of the patient's blood. The PET is accordingly typically performed at a clinic.

In a clinical setting, it may be difficult if not impossible to blend dialysate solutions of different glucose levels to achieve a desired hybrid glucose level. A need therefore exists for a way to readily model hybrid glucose level dialysates.

### SUMMARY

The present disclosure provides an accurate and readily implemented method for modeling blended or hybrid glucose level dialysates. The method analyses each glucose level dialysate component separately and sums or integrates the results. This is done as opposed to actually blending the constituent glucose level dialysates, eliminating the need to manually or automatically blend the dialysates and preventing the possibility of error in blending. An error in blending for example leads to results that predict the patient's response to a glucose level blend that is different than what it is supposed to be.

The inventors have found, using mathematical models, e.g., via a three-pore kinetic modeling, that summing the results of individual dialysates having differing glucose levels yields an overall result that closely approximates the result of a blend of each of the components. For example, results for overall UF, urea Kt/V, cumulative creatinine removed, total carbohydrates (“CHO”) absorbed and total sodium removed showed very good correlation for two 1.5% plus two 2.5% dextrose level fills (corresponding to glucose levels of 1.36% of 2.27%, respectively) versus four 2.0% dextrose level fills. Tests performed and discussed in detail below also showed good correlation for patients with different peritoneal membrane transport types, including high, high average, low average and low patient types. The tests were performed using modeling simulated via a modified three-pore kinetic model discussed in more detail below.

It was also found that the model provides a way to estimate the UF and Kt/V based on a glucose concentration that is cumulative of multiple solution bags with different glucose concentrations, and which is independent of the order of infusion and glucose content of the solutions. It was further found that a linear relationship exists between (i) UF removed and % glucose and (ii) Kt/V and % glucose. Thus, one this linear relationship is learned for the patient, any desired final glucose concentration, not only the ones that could be obtained by mixing readily available solution bags, can be predicted.

It is accordingly an advantage of the present disclosure to provide a method of readily and accurately predicting the results for a dialysis treatment that uses a dialysate blended from different glucose level dialysates without having to actually blend such dialysates.

It is another advantage of the present disclosure to predict the results of a blended PD dialysate therapy for patients having different PD transport characteristics.

It is a further advantage of the present disclosure to develop linear relationships for UF removed and Kt/V versus glucose percentage, such that results for UF removed and Kt/V can be predicted for any final blended glucose level.

It is yet another advantage of the present disclosure to produce a database of the above linear equations, which are accessed using a two dimensional chart, wherein one dimension maps transport status as: high (“H”), high average (“HA”), low average (“LA”), and low (“L”) transport versus a second dimension which maps a particular therapy, e.g., eight hour, four exchange, two liter therapy.

### BRIEF DESCRIPTION OF THE FIGURES

FIG. 1 is a chart illustrating physical characteristics for four patients having different transport statuses that were used in the modeling of the unmixed and mixed solutions.

FIG. 2 shows estimated peritoneal transport parameters for the four patients demonstrated in FIG. 1, such data used in the kinetic modeling of the different dextrose concentrations in combination with the patient data.

FIG. 3 illustrates three solution combinations each modeled using different unmixed dextrose concentrations against like volumes of a mixed or blended dextrose concentration.

FIG. 4 illustrates a comparison of modeled results for net ultrafiltration for one of the combinations shown in FIG. 3 and shown (individual example) for each of the patient transport categories illustrated in FIG. 1.

FIG. 5 illustrates a comparison of modeled results for urea Kt/V for one of the combinations shown in FIG. 3 and shown (individual example) for each of the patient transport categories illustrated in FIG. 1.

FIG. 6 illustrates a correlation between net ultrafiltration and solution concentration for each patient transport category illustrated in FIG. 1.

FIG. 7 illustrates a correlation between urea Kt/V and solution concentration for each patient transport category illustrated in FIG. 1.

FIG. 8A is a schematic flow diagram showing one use of the presently disclosed methodology and corresponding apparatus.

FIG. 8B is a schematic flow diagram showing another use of the presently disclosed methodology and corresponding apparatus.

FIG. 9 is a matrix of stored equations for urea Kt/V and ultrafiltration removed, which are selected based upon a prescribed therapy and the patient's transport status.

### DETAILED DESCRIPTION

The present disclosure addresses a growing need to know the peritoneal dialysis (“PD”) therapy outcomes when at least two different glucose concentrations are mixed to form a new blended solution, which is customized to suit the particular patient. The methodology discussed herein allows customized glucose results to be predicted, leading to a preferred mixture of standard solution, which can be mixed in real time by an automatic peritoneal dialysis (“APD”) machine.

The methodology uses relatively simple, linear equations that relate mixed final solution glucose concentration to therapy outcomes, such as net ultrafiltration (“UF”), weekly urea Kt/V, and creatinine clearance.

In one embodiment, a modified three-pore kinetic model of PD transport was used as the basis for the predictive mathematical model. One suitable modified three-pore kinetic model is described in Rippe B., Sterlin G., and Haraldsson B., Computer Simulations of Peritoneal Fluid Transport in CAPD, Kidney Int. 1991; 40: 315 to 325. Another suitable modified three-pore kinetic model is described in Vonesh E. F. and Rippe B., Net Fluid Adsorption Under Membrane Transport Models of Peritoneal Dialysis, Blood Purif. 1992; 10: 209 to 226, the entire contents of each of which are incorporated herein by reference and relied upon. Matlab™ (version 7.5.0.342, Mathworks Inc.) was used to construct the model.

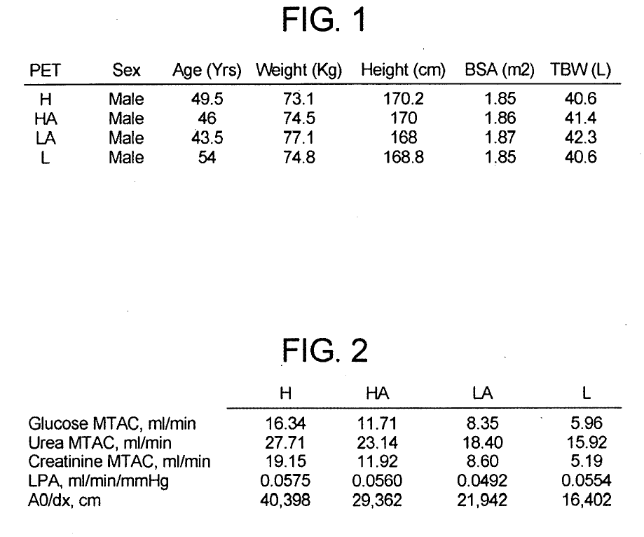

The patient parameters used to illustrate the present method were obtained from data submitted to the assignee of the present disclosure in 1999 by centers around the United States and Canada participating in a national adequacy initiative program. The data were grouped in categories according to the patient's peritoneal transport status as: high (“H”), high average (“HA”), low average (“LA”), and low (“L”) transport statuses. A typical patient for each category was selected as shown in FIGS. 1 and 2. FIG. 1 shows patient physical characteristics. FIG. 2 shows physiological characteristics and kinetic parameters for each patient and corresponding transport category. BSA stands for “body surface area” and TBW stands for “total body water”. MTAC stands for “mass transport area coefficient”. LPA stands for ultrafiltration coefficient. A0/dx stands for unrestricted pore area over unit diffusion distance. As seen in FIG. 2 each clearance value decreased as the transports statuses transition from H to L.

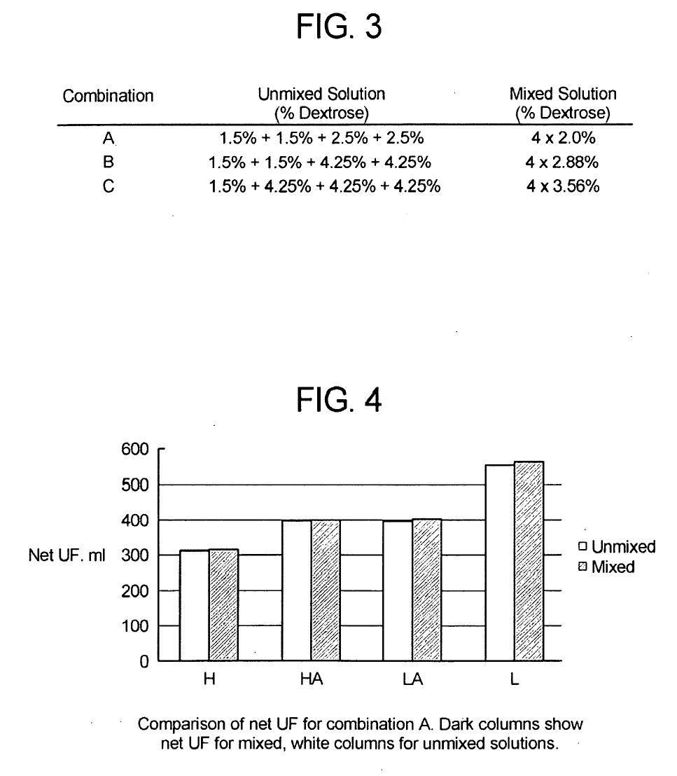

FIG. 3 shows three sets of simulations that were performed with various dextrose concentrations. Combination A pits four unmixed treatments using two each of standard 1.5% and 2.5% dextrose concentration treatments against like volumes of four treatments using a blended 2.0% dextrose concentration. Combination B pits four unmixed treatments using two each of standard 1.5% and 4.25% dextrose concentration treatments against like volumes of four treatments using a blended 2.88% dextrose concentration. Combination C pits four unmixed dextrose concentration treatments using one standard 1.5% and three standard 4.25% dextrose concentration treatments against four treatments using a blended 3.56% dextrose concentration. It is desired to show, and indeed Applicants do show, that the results of the treatments using the unmixed and blended concentrations for each Combination A to C are at least substantially the same.

For each Combination A to C, an 8-hour, 4-exchange therapy was analyzed using 2 liter fills for each unmixed or blended concentration. The difference in the simulated outcomes between unmixed and mixed solution conditions above have been summarized for key parameters such as net UF, urea Kt/V, creatinine clearance, glucose absorbed, and sodium removed. Detailed results are shown in the following Tables, 1A, 1B, 2A, 2B, 3A and 3B. Tables 1A, 1B, 2A, 2B, 3A and 3C show excellent correlation for each combination A to C, for each patient (identified in the tables as Patients 2, 5, 8 and 11) and thus for each patient clearance type H, HA, L, LA, respectively.

Table 1B for Patient 2 (high clearance) shows that when Combination A was modeled in two different combination orders of 1.5% versus 2.5% dextrose concentration combinations, the modeled results for mixed 2.0% concentration matched both unmixed combination orders very closely. Table 2B for Patient 8 (low clearance) shows that when combination B was modeled in two different combination orders of 1.5% versus 4.25% dextrose concentration combinations, the modeled results for mixed 2.88% concentration match both unmixed combination orders vary closely. Table 3B for Patient 5 (high average clearance) shows that when combination C was modeled in two different combination orders of 1.5% versus 4.5% dextrose concentration combinations, the modeled results for mixed 3.56% both unmixed combination orders vary closely.

Tables 1A, 1B, 2A, 2B, 3A and 3B

TABLE 1A
for Combination A - Results (1.5% and 2.5% 1:1)

| | | | | | | | | Cum | | |
|---|---|---|---|---|---|---|---|---|---|---|
| | | | | | | | Cum | Weekly | Total | |
| | | | | | | Net UF | Weekly | Ccr | COH | Total Sodium |
| | | | | | | per Day | Urea | (L/week/ | Absorbed | Removed |
| | | PatientID | BSA | PET | Period | (ml) | Kt/V | 1.73) | (g) | (mmol) |
| Unmixed | Unmixed (1.5% | 2 | 1.84 | H | NIGHT | 310 | 1.166 | 37.694 | 85.78 | 43.03 |
| vs. | 1.5% 2.5% | 8 | 1.85 | L | NIGHT | 553 | 0.918 | 16.408 | 41.76 | 50.20 |
| Mixed | 2.5%) | 5 | 1.86 | HA | NIGHT | 397 | 1.071 | 28.768 | 69.71 | 44.88 |
| | | 11 | 1.87 | LA | NIGHT | 398 | 0.940 | 22.989 | 54.89 | 40.53 |
| | Mixed (2.0%) | 2 | 1.84 | H | NIGHT | 313 | 1.168 | 37.709 | 85.90 | 43.25 |
| | | 8 | 1.85 | L | NIGHT | 560 | 0.920 | 16.428 | 41.76 | 50.64 |
| | | 5 | 1.86 | HA | NIGHT | 401 | 1.073 | 28.785 | 69.81 | 45.18 |
| | | 11 | 1.87 | LA | NIGHT | 403 | 0.942 | 23.005 | 54.92 | 40.83 |

| | | | | | | | | | |
|---|---|---|---|---|---|---|---|---|---|
| Alternate Order (1.5% 2.5% | 2 | 1.84 | H | NIGHT | 314 | 1.169 | 37.705 | 85.46 | 43.14 |
| 1.5% 2.5%) | 8 | 1.85 | L | NIGHT | 560 | 0.921 | 16.428 | 41.05 | 50.44 |
| | 5 | 1.86 | HA | NIGHT | 402 | 1.075 | 28.780 | 69.25 | 45.04 |
| | 11 | 1.87 | LA | NIGHT | 404 | 0.943 | 23.001 | 54.32 | 40.70 |

TABLE 1B
for Combination A - Breakout Box for Patient ID 2 for Easier Comparison

| | | | | | | | Cum | | |
|---|---|---|---|---|---|---|---|---|---|
| | | | | | | Cum | Weekly | Total | Total |
| | | | | | Net UF | Weekly | Ccr | COH | Sodium |
| | | | | | per Day | Urea | (L/week/ | Absorbed | Removed |
| | PatientID | BSA | PET | Period | (ml) | Kt/V | 1.73) | (g) | (mmol) |
| Unmixed (1.5% 1.5% | 2 | 1.84 | H | NIGHT | 310 | 1.166 | 37.694 | 85.78 | 43.03 |
| 2.5% 2.5%) | | | | | | | | | |
| Mixed (2.0%) | 2 | 1.84 | H | NIGHT | 313 | 1.168 | 37.709 | 85.90 | 43.25 |
| Alternate Order (1.5% | 2 | 1.84 | H | NIGHT | 314 | 1.169 | 37.705 | 85.46 | 43.14 |
| 2.5% 1.5% 2.5%) | | | | | | | | | |

TABLE 2A
for Combination B - Results (1.5% and 4.25% 1:1)

| | | | | | | | | Cum | | |
|---|---|---|---|---|---|---|---|---|---|---|
| | | | | | | | Cum | Weekly | Total | |
| | | | | | | Net UF | Weekly | Ccr | COH | Total Sodium |
| | | Patient | | | | per Day | Urea | (L/week/ | Absorbed | Removed |
| | | ID | BSA | PET | Period | (ml) | Kt/V | 1.73) | (g) | (mmol) |
| Unmixed | Unmixed | 2 | 1.84 | H | NIGHT | 697 | 1.212 | 38.981 | 121.74 | 79.48 |
| vs. | (1.5% | 8 | 1.85 | L | NIGHT | 1029 | 0.961 | 17.516 | 55.77 | 91.57 |
| Mixed | 1.5% | 5 | 1.86 | HA | NIGHT | 816 | 1.116 | 29.897 | 97.34 | 82.94 |
| | 4.25% | 11 | 1.87 | LA | NIGHT | 800 | 0.977 | 23.959 | 75.85 | 75.83 |
| | 4.25%) | | | | | | | | | |
| | Mixed | 2 | 1.84 | H | NIGHT | 709 | 1.219 | 39.047 | 123.30 | 80.80 |
| | (2.875%) | 8 | 1.85 | L | NIGHT | 1056 | 0.969 | 17.571 | 57.20 | 93.90 |
| | | 5 | 1.86 | HA | NIGHT | 834 | 1.123 | 29.959 | 98.98 | 84.63 |
| | | 11 | 1.87 | LA | NIGHT | 819 | 0.983 | 24.011 | 77.25 | 77.48 |

| | | | | | | | | | |
|---|---|---|---|---|---|---|---|---|---|
| Alternate Order (1.5% | 2 | 1.84 | H | NIGHT | 709 | 1.223 | 39.012 | 120.85 | 79.84 |
| 4.25% 4.25% 1.5%) | 8 | 1.85 | L | NIGHT | 1047 | 0.972 | 17.571 | 53.92 | 92.27 |
| | 5 | 1.86 | HA | NIGHT | 831 | 1.126 | 29.928 | 96.11 | 83.42 |
| | 11 | 1.87 | LA | NIGHT | 814 | 0.986 | 23.992 | 74.35 | 76.31 |

TABLE 2B
for Combination B - Breakout Box for Patient ID 8 for Easier Comparison

| | | | | | | | | Total | |
|---|---|---|---|---|---|---|---|---|---|
| | | | | | Net UF | Cum Weekly | Cum Weekly | COH | Total Sodium |
| | | | | | per Day | Urea | Ccr (L/week/ | Absorbed | Removed |
| | PatientID | BSA | PET | Period | (ml) | Kt/V | 1.73) | (g) | (mmol) |
| Unmixed (1.5% | 8 | 1.85 | L | NIGHT | 1029 | 0.961 | 17.516 | 55.77 | 91.57 |
| 1.5% 4.25% | | | | | | | | | |
| 4.25%) | | | | | | | | | |
| Mixed | 8 | 1.85 | L | NIGHT | 1056 | 0.969 | 17.571 | 57.20 | 93.90 |
| (2.875%) | | | | | | | | | |
| Alternate Order | 8 | 1.85 | L | NIGHT | 1047 | 0.972 | 17.571 | 53.92 | 92.27 |
| (1.5% 4.25% | | | | | | | | | |
| 4.25% 1.5%) | | | | | | | | | |

TABLE 3A
for Combination C - Results (1.5% and 4.25% 3:1)

| | | | | | | | | Cum | | |
|---|---|---|---|---|---|---|---|---|---|---|
| | | | | | | | Cum | Weekly | Total | Total |
| | | | | | | Net UF | Weekly | Ccr | COH | Sodium |
| | | | | | | per Day | Urea | (L/week/ | Absorbed | Removed |
| | | PatientID | BSA | PET | Period | (ml) | Kt/V | 1.73) | (g) | (mmol) |
| Unmixed | Unmixed | 2 | 1.84 | H | NIGHT | 1009 | 1.255 | 40.021 | 150.18 | 108.63 |
| vs. | (1.5% | 8 | 1.85 | L | NIGHT | 1417 | 1.002 | 18.418 | 66.60 | 125.02 |
| Mixed | 4.25% | 5 | 1.86 | HA | NIGHT | 1157 | 1.158 | 30.810 | 119.15 | 113.52 |
| | 4.25% | 11 | 1.87 | LA | NIGHT | 1126 | 1.012 | 24.744 | 92.25 | 104.23 |
| | 4.25%) | | | | | | | | | |
| | Mixed | 2 | 1.84 | H | NIGHT | 1017 | 1.259 | 40.074 | 151.49 | 109.62 |
| | (3.5625%) | 8 | 1.85 | L | NIGHT | 1436 | 1.007 | 18.467 | 67.92 | 126.72 |
| | | 5 | 1.86 | HA | NIGHT | 1168 | 1.162 | 30.865 | 120.57 | 114.76 |
| | | 11 | 1.87 | LA | NIGHT | 1139 | 1.015 | 24.791 | 93.51 | 105.45 |

| | | | | | | | | | |
|---|---|---|---|---|---|---|---|---|---|
| Alternate Order (4.25% | 2 | 1.84 | H | NIGHT | 1021 | 1.266 | 40.074 | 149.36 | 109.08 |
| 4.25% 4.25% 1.5%) | 8 | 1.85 | L | NIGHT | 1436 | 1.014 | 18.515 | 64.86 | 125.87 |
| | 5 | 1.86 | HA | NIGHT | 1171 | 1.169 | 30.875 | 118.01 | 114.11 |
| | 11 | 1.87 | LA | NIGHT | 1141 | 1.021 | 24.810 | 90.84 | 104.82 |

TABLE 3B
for Combination C - Breakout Box for Patient ID 5 for Easier Comparison

| | | | | | | | Cum | | |
|---|---|---|---|---|---|---|---|---|---|
| | | | | | | Cum | Weekly | Total | Total |
| | | | | | Net UF | Weekly | Ccr | COH | Sodium |
| | | | | | per Day | Urea | (L/week/ | Absorbed | Removed |
| | PatientID | BSA | PET | Period | (ml) | Kt/V | 1.73) | (g) | (mmol) |
| Unmixed (1.5% 4.25% | 5 | 1.86 | HA | NIGHT | 1157 | 1.158 | 30.810 | 119.15 | 113.52 |
| 4.25% 4.25%) | | | | | | | | | |
| Mixed (3.5625%) | 5 | 1.86 | HA | NIGHT | 1168 | 1.162 | 30.865 | 120.57 | 114.76 |
| Alternate Order (4.25% | 5 | 1.86 | HA | NIGHT | 1171 | 1.169 | 30.875 | 118.01 | 114.11 |
| 4.25% 4.25% 1.5%) | | | | | | | | | |

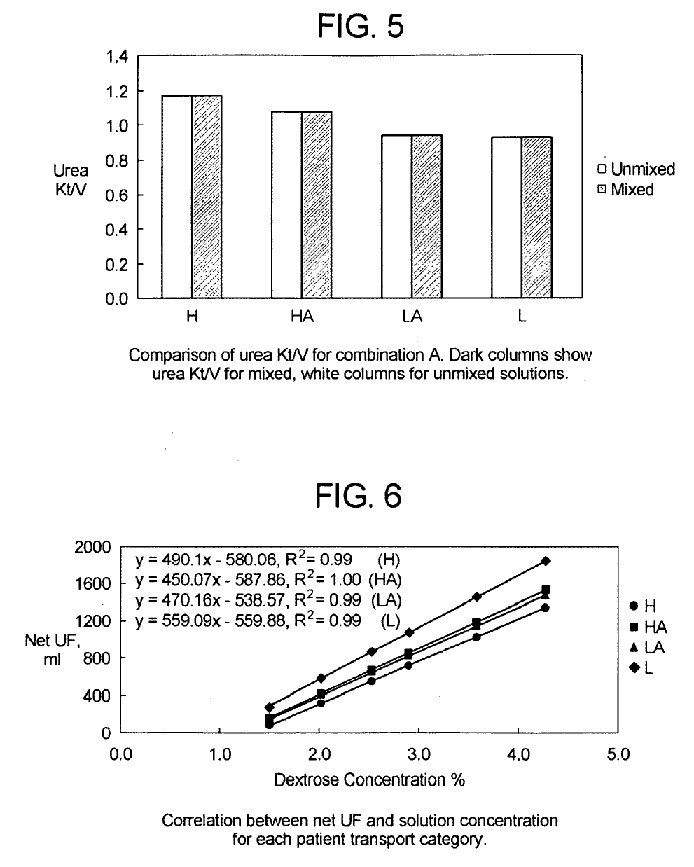

FIGS. 4 and 5 illustrate graphically the simulated and combined results for net UF and urea Kt/V, respectively, for combination A. As is illustrated, both parameters indicate that infusion of 1.5% and 2.5% dialysate solutions separately (2×1.5%+2×2.5%) and in mixed form (4×2.0%) result in virtually equivalent outcomes. The results demonstrate that (1) it is possible to predict outcomes of therapies in which mixed solutions are used and (2) that the outcomes are virtually the same for mixed and unmixed solutions. These predicted results are expected to translate well to actual results, such that a patient can run treatments using different unmixed solutions to generate actual data that will correspond to how the patient will perform using corresponding mixed solutions. Thus, once the present method is proven for a given patient with a one-to-one comparison between mixed and unmixed solutions, the method can then be used to predict outcomes of any final solution concentration for that patient.

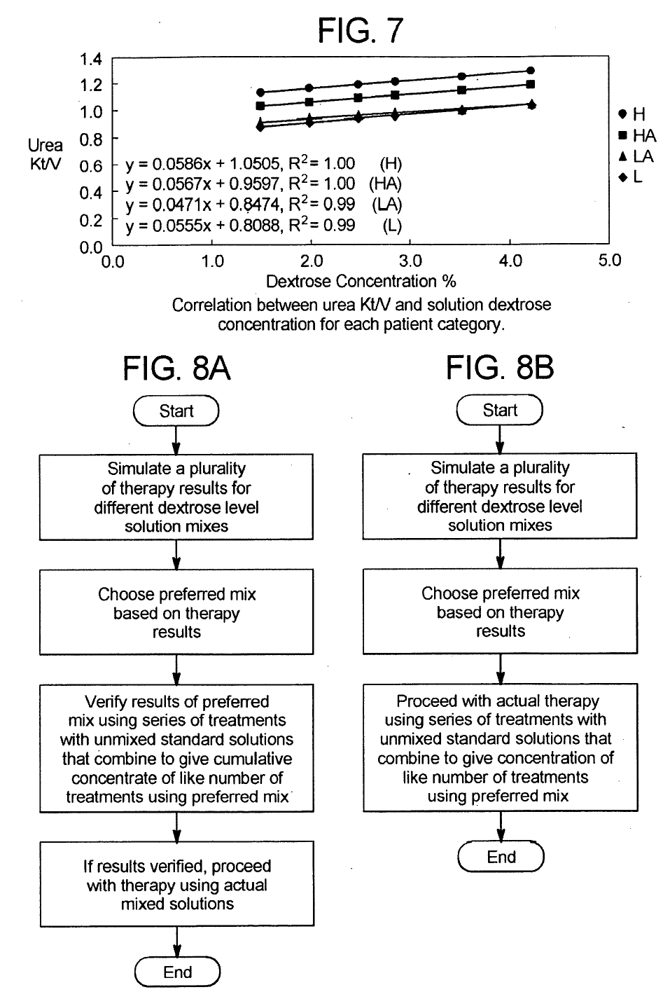

FIGS. 6 and 7 illustrate another useful feature of the methodology of the present discourse, namely, that there are linear relationships between the outcomes and the mixed dextrose concentrations. Two examples are shown respectively in FIGS. 6 and 7 for net UF and weekly urea Kt/V. The linear relationships lead to equations that allow for the estimation of outcomes based on any dextrose concentration within the 1.5% to 4.25% standardized dextrose range. The relationships may also be mathematically extended outside this range, or within a larger range of unmixed solutions. It is contemplated to store these relationships on a computer memory for recall and use. One significant benefit is that knowing the relatively simple linear relationships, running more complicated kinetic models for the patient, for different final mixed glucose levels will not be necessary.

It should be understood that the equations shown in FIGS. 6 and 7 are true for and particular to the conditions of the therapy for which the simulations were performed, e.g., an eight hour, four exchange, two liter therapy. If the patient's therapy is changed, new models should be generated.

The above simulations demonstrate that PD therapies conducted by infusing a mixture of readily available dextrose concentrations are equivalent to infusing each conventional solution one at a time as long as the cumulative dextrose concentration is the same. It is also shown that a linear relationship exists between the dextrose concentration and the outcomes.

The ability to demonstrate such equivalence is important in a variety of ways. First, the methodology provides a useful way to support regulatory claims in the area of solution mixing. Second, as seen in FIG. 8A, the simulated outcomes can be used to select a “favorite or “optimal”, or “customized” mixed solution, whose simulated results are then verified using an actual test using unmixed standard dextrose solutions that provide a cumulative concentration that would have been obtained using the preferred mixed solution, assuming that the final desired can be obtained by a combination of the existing solutions. Thus, no actual mixing has to take place until the mixed solution is approved for use. Third, as seen in FIG. 8B, the patient continues to use the unmixed solutions in the order needed to simulate the mixed solution for actual therapies. Here, actual mixing never takes place. In both the applications of FIG. 8A, it is contemplated to perform the simulation steps using either a single mixed concentration or a combination of unmixed concentrations. That is, if it is easier to do so, a single mixed concentration can be used for the modeling. The benefit of not having to actually mix the standard concentrations is then achieved in the actual verifying step of FIG. 8A or the actual therapy performance step of FIG. 8B.

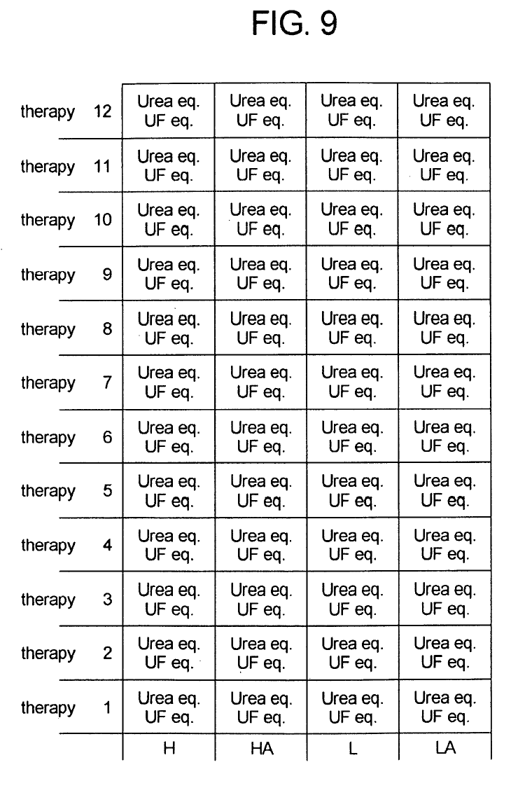

Another key aspect of the present method is the generation of the linear simple relationships for UF and urea Kt/V, which allows any suitable glucose concentrations to be predicted. FIG. 9 illustrates a two-dimensional matrix for relationships, such as those shown in FIGS. 6 and 7. The x-axis of the matrix maps transport status as: high (“H”), high average (“HA”), low average (“LA”), and low (“L”) transport. The y-axis of the matrix maps a type of therapy, e.g., an (i) eight hour, four exchange, two liter therapy versus (ii) a nine hour, five exchange, 1.75 liter therapy. Each intersection includes a corresponding equation for UF and urea Kt/V versus glucose level. The matrix could include equations based on single patient's input data for the particular transport status or averaged data (or middle of a range data) for the particular transport status. In any case, the matrix can be used to provide some indication to a new patient who falls into a particular transport status, what the results would be for a particular therapy using a particular glucose level dialysate, that glucose level being a standard or blended glucose level. If a blended glucose level, then the present method contemplates provided a prescription using standard glucose level dialysates that combine to mimic or equal the blended glucose level treatment.

It should be appreciated that the disclosed methodology and resulting apparatus can be extended to non-glucose based solutions, glucose-based solutions with lower sodium concentrations, or bi-modal solutions.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for predicting results of a peritoneal dialysis therapy for a patient using dialysate blended from a plurality of dialysates having different glucose levels, said method comprising:
determining a therapy outcome parameter for the patient using a first one of the dialysates having a first one of the glucose levels;
determining the therapy outcome parameter for the patient using a second one of the dialysates having a second one of the glucose levels;
combining the therapy outcome parameters obtained from use of the first and second contact dialysates to form a combined therapy outcome parameter; and
assuming the combined therapy outcome parameter to be a totaled therapy outcome parameter using the dialysate blended from the first and second dialysates.

2. The method of claim 1, which includes assuming the combined therapy outcome parameter to be for a volume totaled from a volume used for the first dialysate and a volume used for the second dialysate.

3. The method of claim 1, wherein combining the therapy outcome parameters includes summing the therapy outcome parameters.

4. The method of claim 1, wherein combining the therapy outcome parameters includes at least one of: (i) net UF removed; (ii) cumulative urea removed; (iii) cumulative creatinine removed; (iv) total carbohydrate absorbed; and (v) total sodium removed.

5. The method of claim 1, wherein determining the therapy outcome parameter for at least one of the first and second dialysates includes using a mathematical model.

6. The method of claim 1, wherein the first and second glucose levels are one of 1.36%, 2.27% and 3.86%.

7. The method of claim 1, wherein using one of the first and second dialysates includes using multiple fills of the dialysate.

8. The method of claim 7, wherein determining the therapy outcome parameter using multiple fills of the first or second dialysates includes combining the therapy outcome parameters of the multiple fills.

9. The method of claim 8, wherein combining the therapy outcome parameters of the multiple fills includes summing the therapy outcome parameters.

10. The method of claim 1, wherein assuming the combined therapy outcome parameter includes making the assumption for a particular type of patient transport status of the patient.

11. The method of claim 1, wherein determining the therapy outcome parameter for at least one of the first and second dialysates includes inputting at least one value based on a patient transport status belonging to the patient.

12. The method of claim 11, wherein the at least one inputted value is selected from the group consisting of: glucose mass transport area coefficient (“MTAC”), urea MTAC, creatinine MTAC, ultrafiltration coefficient (“LPA”), and unrestricted pore area over unit diffusion distance (“Ao/dx”).

13. The method of claim 1, which includes determining the therapy outcome parameter for the patient using a third one of the dialysates having a third one of the glucose levels and combining the therapy outcome parameters from use of the first, second and third dialysates to form the combined therapy outcome.

14. A computer readable medium modified to perform the method of claim 1.

15. A method for predicting results of a peritoneal dialysis therapy for a patient using dialysate blended from a plurality of dialysates having different glucose levels, said method comprising:
determining an ultrafiltration removed for the patient using a first one of the dialysates having a first one of the glucose levels;
determining the ultrafiltration removed for the patient using a second one of the dialysates having a second one of the glucose levels;
adding the ultrafiltration removed obtained from use of the first and second dialysates to form a combined ultrafiltration removed; and
assuming the combined ultrafiltration removed to be a blended ultrafiltration removed using a dialysate actually blended from the first and second dialysates.

16. A method for predicting results of a peritoneal dialysis therapy for a patient using dialysate blended from a plurality of dialysates having different glucose levels, said method comprising:
determining a urea Kt/V for the patient using a first one of the dialysates having a first one of the glucose levels;
determining the urea Kt/V for the patient using a second one of the dialysates having a second one of the glucose levels;
determining a combined urea Kt/V obtained from use of the first and second dialysates; and
assuming the combined urea Kt/V to be a blended urea Kt/V using a dialysate actually blended from the first and second dialysates.

17. A method for predicting results of a peritoneal dialysis therapy for a patient using dialysate blended from a plurality of dialysates having different glucose levels, said method comprising:
determining a creatinine removed for the patient using a first one of the dialysates having a first one of the glucose levels;
determining the creatinine removed for the patient using a second one of the dialysates having a second one of the glucose levels;
determining a combined creatinine removed from use of the first and second dialysates; and
assuming the combined creatinine removed to be a blended creatinine removed using a dialysate actually blended from the first and second dialysates.

18. A method of selecting a dialysis solution for a patient comprising:
predicting results of a plurality of patient therapy outcomes for a plurality of different mixed dextrose level dialysis solutions;
selecting one of the mixed dextrose level solutions for a patient based on the results; and
verifying the results by prescribing a number of therapies using different unmixed dextrose level solutions that combine to simulate a like cumulative concentration using the selected mixed dextrose level solution.

19. The method of claim 18, wherein predicting results of the plurality of patient therapy outcomes for the plurality of different mixed dextrose level dialysis solutions includes using unmixed dextrose level solutions that combine to simulate a like cumulative concentration using the particular mixed dextrose level solution.

20. The method of claim 18, wherein predicting results of the plurality of patient therapy outcomes for the plurality of different mixed dextrose level dialysis solutions includes using a single mixed dextrose level concentration.

21. A method for peritoneal dialysis treatment comprising:
predicting results of a plurality of patient therapy outcomes for a plurality of different mixed dextrose level dialysis solutions;
selecting one of the mixed dextrose level solutions for a patient based on the results; and
performing at least one therapy using different unmixed dextrose level solutions that combine to simulate a like cumulative concentration that would be achieved using the selected mixed dextrose level solution.

22. The method of claim 21, wherein predicting results of the plurality of patient therapy outcomes for the plurality of different mixed dextrose level dialysis solutions includes using unmixed dextrose level solutions that combine to simulate a like cumulative concentration using the particular mixed dextrose level solution.

23. The method of claim 21, wherein predicting results of the plurality of patient therapy outcomes for the plurality of different mixed dextrose level dialysis solutions includes using a single mixed dextrose level concentration.

24. A method for peritoneal dialysis treatment comprising:
determining a relationship for patient ultrafiltration (“UF”) or patient Kt/V based on results for a first glucose level dialysate and a second glucose level dialysate; and
using the relationship to predict UF or patient urea Kt/V for a third glucose level dialysate.

25. The method of claim 24, which includes predicting the ultrafiltration (“UF”) or patient urea Kt/V results for the first and second glucose level dialysates.

26. The method of claim 24, which includes prescribing a therapy that uses the third glucose level dialysate, the third glucose level being a non-standard glucose level.

27. The method of claim 26, wherein prescribing the therapy includes using a combination of standard glucose level dialysates that combine to mimic the non-standard third glucose level dialysate.

28. The method of claim 24, wherein the third glucose level is between the first and second glucose levels.

29. The method of claim 24, wherein the relationship is linear.
